(19) United States
(12) Patent Application Publication
(10) Pub. No.: US 2021/0228832 A1
(43) Pub. Date: Jul. 29, 2021

(54) CONTINUOUS POSITIVE AIRWAY PRESSURE DEVICE FOR NEONATES

(71) Applicant: D-Rev: Design For the Other Ninety Percent, San Francisco, CA (US)

(72) Inventors: Robin PARRISH, Oakland, CA (US); Elise DEVRIES, San Francisco, CA (US)

(21) Appl. No.: 17/228,521

(22) Filed: Apr. 12, 2021

Related U.S. Application Data
(63) Continuation of application No. PCT/US19/55891, filed on Oct. 11, 2019.
(60) Provisional application No. 62/836,993, filed on Apr. 22, 2019.
(60) Provisional application No. 62/748,274, filed on Oct. 19, 2018.

(51) Int. Cl. A61M 16/16; A61M 16/00; A61M 16/04

(52) U.S. Cl. CPC A61M 16/161; A61M 2016/1025; A61M 16/0066; A61M 16/049; A61M 16/0003; A61M 2205/3368; A61M 2205/3334; A61M 2205/502; A61M 2205/583; A61M 2205/581; A61M 2230/42; A61M 2230/50; A61M 2230/202; A61M 2230/205; A61M 16/0051

(57) ABSTRACT

A continuous positive airway pressure system comprises an inspiratory portion, an expiratory portion, and a controller. The inspiratory portion is coupled to a patient interface to provide an airflow with positive pressure to a patient. The inspiratory portion includes a first sensor to measure a pressure and/or a flow rate of the airflow at the inspiratory portion. The expiratory portion is coupled to the patient interface to receive air exhaled from the patient. The expiratory portion includes a second sensor for measuring a pressure and/or a flow rate of the air exhaled at the expiratory portion. The controller (i) determines a pressure at the patient interface based on the measured pressures and/or the flow rates of the airflow at the inspiratory portion and the air exhaled at the expiratory portion and (ii) modifies the airflow provided by the inspiratory portion based on the determined pressure.

### CROSS-REFERENCE

This application is a continuation of PCT Application No. PCT/US19/55891, filed Oct. 11, 2019; which claims the benefit of U.S. Provisional Application Nos. 62/836,993, filed Apr. 22, 2019; and 62/748,274, filed Oct. 19, 2018; all of which are incorporated herein by reference in their entirety.

### BACKGROUND

The present disclosure relates generally to the respiratory support field and more specifically to an improved continuous positive airway pressure (CPAP) device designed for neonates with Respiratory Distress Syndrome (RDS).

Clinicians characterize respiratory distress (RD) in newborns as difficulty breathing and poor oxygen saturation of varying severity, with Respiratory Distress Syndrome (RDS) leading to the most severe cases. RDS is most common in premature infants; each year, roughly 3.2 million globally are thought to suffer from RD that CPAP could treat. The main goal of treatment is to maintain appropriate oxygenation (measured as SpO2).

Although the mortality rate of RDS without treatment is nearly 100%, it is only 2% when appropriately treated. However, in low-income countries with poor health outcomes, the mortality rate remains as high as 75%. While multiple factors contribute to this high mortality rate, the significant factors involved in current CPAP therapy failure are the need for consistent monitoring, as current solutions only work when the nasal prongs are fully occluded within the infant's nares, and the high baby:nurse ratio found in low-income countries that does not allow nurses to provide sufficient oversight to all neonates.

Accordingly, improved systems, devices, and methods for neonatal CPAP are desired.

Reference which may be relevant to the disclosure herein may include U.S. Pat. Nos. 4,340,044, 5,535,738, 5,694,923, 5,701,883, 5,794,615, 6,299,581, 6,360,741, 6,694,976, 7,284,554, 8,256,417, 8,261,742, 8,967,144, 9,302,066, 9,399,109, and 9,999,742 and U.S. Publications US2004226562, US2005098179, US2006213518, US2009007911, US2010319691, US2013228180, US2016022954, and US2016199607.

### SUMMARY

Aspects of the present disclosure provide continuous positive airway pressure (CPAP) systems. An exemplary CPAP system may comprise an inspiratory module, which may comprise a first inlet for a compressed oxygen source, a second inlet for an ambient air source, a blender coupled to the first and second inlets to blend compressed oxygen and ambient air from the first and second inlets, respectively, and a blower coupled to the blender and configured to generate an airflow directed to a patient from the blended air.

The inspiratory module may include further features. The inspiratory module may further comprise an outlet to couple to a patient interface and direct the airflow thereto. The patient interface may comprise one or more of a nasal cannula, a nasal prong, a nasopharyngeal tube, an esophageal tube, a mouth piece, or a face mask. The inspiratory module may further comprise a heater to provide heat to the airflow generated by the blower. The inspiratory module may further comprise a temperature sensor to measure a temperature of the airflow. The inspiratory module may further comprise a controller configured to control the amount of heat provided to the airflow by the heater based on the measured temperature. The inspiratory module may further comprise a humidifier to provide humidity to the airflow generated by the blower. The inspiratory module may further comprise a humidity sensor to measure a humidity of the airflow. The inspiratory module may further comprise a controller configured to control the amount of humidity provided to the airflow by the humidifier based on the measured humidity.

The inspiratory module may further comprise one or more sensors. The inspiratory module may further comprise a pressure sensor to measure a pressure of the airflow generated by the blower. The inspiratory module may further comprise a controller configured to control the blower based on the measured pressure. The inspiratory module may further comprise a flow rate sensor to measure a flow rate of the airflow generated by the blower. The inspiratory module may further comprise a controller configured to control the blower based on the measured flow rate. The inspiratory module may further comprise an oxygen sensor to measure an oxygenation of the blended air. The inspiratory module may further comprise a controller configured to control a ratio of compressed air provided through the first inlet to ambient air provided by the second inlet based on the measured oxygenation.

The inspiratory module may comprise two or more of (i) a compressed oxygen sensor to measure one or more of a flow rate or pressure from the compressed air from the first inlet, (ii) an ambient air sensor to measure one or more of a flow rate or pressure from the ambient air from the second inlet, or (iii) a blended air sensor to measure one or more of a flow rate or pressure from the blended air from the blender. The inspiratory module may further comprise a controller coupled to the two or more of (i) the compressed oxygen sensor, (ii) the ambient air sensor, or (iii) the blended air sensor to determine an oxygenation of the blended air based on the flow rates or pressures from two or more of (i) the compressed air, (ii) the ambient air, and (iii) the blended air. The controller may be configured to control a ratio of compressed air provided through the first inlet to ambient air provided by the second inlet based on the determined oxygenation.

The compressed air source may comprise one or more of wall oxygen, an oxygen tank, or a compressed oxygen line.

According to further aspects of the present disclosure, an exemplary continuous positive airway pressure (CPAP) system may comprise an inspiratory module, which may comprise a first inlet for a compressed oxygen source, a second inlet for an ambient air source, a blender coupled to the first and second inlets to blend compressed oxygen and ambient air from the first and second inlets, respectively, one or more sensors to determine an oxygenation of the blended air, and a controller configured to control a ratio of the compressed air provided through the first inlet to the ambient air provided by the second inlet based on the determined oxygenation. The ratio may be controlled to maintain a desired range of oxygen concentration in the blended air.

The inspiratory module may include further features. The inspiratory module may further comprise a heater to provide heat to the blended air. The inspiratory module may further comprise a temperature sensor to measure a temperature of the blended air. The inspiratory module may further comprise a controller configured to control the amount of heat provided to the blended air by the heater based on the measured temperature. The inspiratory module may further comprise a humidifier to provide humidity to the blended air. The inspiratory module may further comprise a humidity sensor to measure a humidity of the blended air. The inspiratory module may further comprise a controller configured to control the amount of humidity provided to the blended air by the humidifier based on the measured humidity.

The compressed air source may comprise one or more of wall oxygen, an oxygen tank, an oxygen concentrator, or a compressed oxygen line.

The one or more sensors may comprise an oxygen sensor.

The one or more sensors may comprise two or more of (i) a compressed oxygen sensor to measure one or more of a flow rate or pressure from the compressed air from the first inlet, (ii) an ambient air sensor to measure one or more of a flow rate or pressure from the ambient air from the second inlet, or (iii) a blended air sensor to measure one or more of a flow rate or pressure from the blended air from the blender. The inspiratory module may further comprise a controller coupled to the two or more of the (i) compressed oxygen sensor, (ii) the ambient air sensor, and (iii) the blended air sensor to determine an oxygenation of the blended air based on the flow rates or pressures from two or more of (i) the compressed air, (ii) the ambient air, and (iii) the blended air.

Aspects of the present disclosure also provide methods of generating an airflow for continuous positive airway pressure therapy. Ambient air and compressed oxygen may be provided to a blender to generate blended air. An airflow of the blended air may be generated with a blower. An oxygenation of the blended air may be determined. A ratio of the compressed oxygen to the ambient air provided to the blender may be determined based on the determined oxygenation. The ratio may be controlled to maintain a desired range of oxygen concentration in the blended air.

The blended air may be heated. The temperature of the blended air may be measured, and the amount of heat provided to the blended air may be controlled based on the measured temperature.

The blended air may be humidified. The humidity of the blended air may be measured, and the humidity provided to the blended air may be controlled based on the measured humidity.

One or more of a flow rate or a pressure of the airflow generated by the blower controlling one or more of a compressed air source may be measured, and compressed air may be provided to the blender, the blender, or the blower in response.

The compressed air may be provided from one or more of wall oxygen, an oxygen tank, an oxygen concentrator, or a compressed oxygen line.

The oxygenation of the blended air may be determined with an oxygenation sensor.

The of the blended air may be determined based on one or more of a flow rate or pressure from the compressed oxygen, one or more of a flow rate or pressure from the ambient air, and one or more of a flow rate or pressure from the blended air.

According to further aspects of the present disclosure, an exemplary continuous positive airway pressure (CPAP) system may comprise an inspiratory portion, an expiratory portion, and a controller. The inspiratory portion may be coupled to a patient interface to provide an airflow with positive pressure to a patient through the patient interface. The inspiratory portion may include a first sensor to measure one or more of a pressure or a flow rate of the airflow at the inspiratory portion. The expiratory portion may be coupled to the patient interface to receive air exhaled from the patient. The expiratory portion may include a second sensor for measuring one or more of a pressure or a flow rate of the air exhaled at the expiratory portion. The controller may be configured to (i) determine a pressure at the patient interface based on the measured one or more of the pressure or the flow rate of the airflow at the inspiratory portion and the measured one or more of the pressure or the flow rate of the air exhaled at the expiratory portion and (ii) modify the airflow provided by the inspiratory portion based on the determined pressure at the patient interface.

The inspiratory portion may include further features. The inspiratory portion may comprise a blower to generate the airflow. The controller may be configured to modify the airflow by regulating a speed of the blower. The inspiratory portion may comprise a heater to provide heat to the airflow and a temperature sensor to measure a temperature of the airflow. The controller may be configured to control the amount of heat provided to the airflow by the heater based on the measured temperature. The inspiratory portion may comprise a humidifier to provide humidity to the airflow and a humidity sensor to measure humidity of the airflow. The controller may be configured to control the amount of humidity provided to the airflow by the humidifier based on the measured humidity. The inspiratory portion may comprise a blender to blend ambient air with compressed oxygen and one or more sensors to determine oxygenation of the airflow. The controller may be configured to control a ratio of compressed air to ambient air based on the determined oxygenation. The inspiratory portion may further comprise one or more of a blower to generate the airflow, a heater for the airflow, a humidifier for the airflow, or a blender to blend ambient air with compressed air. The inspiratory portion may further comprise one or more of a flow rate sensor, a pressure sensor, a temperature sensor, a humidity sensor, or an oxygenation sensor. The controller may be configured to increase the airflow if the determined pressure has decreased.

The inspiratory portion may be removably coupled to the patient interface.

The inspiratory portion may be a standalone module.

The expiratory portion may further comprise an air bubbler for the air exhaled.

The expiratory portion may be removably coupled to the patient interface.

The expiratory portion may be a standalone module.

The user interface may be coupled to the controller. The user interface may comprise a visual display. The visual display may be configured to display one or more of the measured pressure of the airflow at the inspiratory portion, the measured flow rate of the airflow at the inspiratory portion, the measured pressure of the airflow at the expiratory portion, the measured flow rate of the airflow at the expiratory portion, the determined pressure at the patient interface, or a calibration status of the system. The visual display may be further configured to display one or more of a measured temperature, a measured humidity, or a measured oxygenation level of the airflow at the inspiratory portion. The visual display may be configured to provide a visual alert for a user or the patient. The visual alert may comprise a captioned alert. The user interface may be configured to provide one or more of a visual alert or an audio alert for a user or the patient. The one or more of the visual alert or the audio alert may indicate one or more of a low inspiratory air pressure, a high inspiratory air pressure, a low inspiratory oxygenation level, a high inspiratory oxygenation level, a low inspiratory air temperature, a high inspiratory air temperature, a battery level, or a system error.

The patient interface may comprise one or more of a nasal cannula, a nasal prong, a nasopharyngeal tube, an esophageal tube, a mouth piece, or a face mask.

According to further aspects of the present disclosure, an exemplary method of generating an airflow for continuous positive airway pressure therapy may be provided. One or more of a pressure or a flow rate of an airflow generated at an inspiratory portion of a continuous positive airway pressure (CPAP) system may be measured. One or more of a pressure or a flow rate of air exhaled and received at an expiratory portion of the CPAP system may be measured. A pressure at a patient interface coupled to the CPAP system may be determined based on the measured one or more of the pressure or the flow rate of the airflow at the inspiratory portion and the measured one or more of the pressure or the flow rate of the air exhaled at the expiratory portion. The airflow provided by the inspiratory portion may be modified based on the determined pressure at the patient interface.

The airflow may be modified in many ways. The airflow may be modified by regulating a speed of a blower of the inspiratory portion of the CPAP system generating the airflow. The airflow may be modified by increasing the airflow if the determined pressure has decreased.

The temperature of the airflow may be measured and an amount of heat provided to the airflow by a heater may be controlled in response.

The humidity of the airflow may be measured and an amount of humidity provided to the airflow by a humidifier may be controlled in response.

The oxygenation of the airflow may be determined and a ratio of compressed oxygen to ambient air blended by a blender of the inspiratory portion of the CPAP system may be controlled based on the determined oxygenation.

The airflow may be directed to a patient through a patient interface coupled to the CPAP system.

The method may comprise displaying many different parameters. One or more of the measured pressure of the airflow at the inspiratory portion, the measured flow rate of the airflow at the inspiratory portion, the measured pressure of the airflow at the expiratory portion, the measured flow rate of the airflow at the expiratory portion, the determined pressure at the patient interface, or a calibration status of the system may be displayed. One or more of a measured temperature, a measured humidity, or a measured oxygenation level of the airflow at the inspiratory portion may be displayed.

The method may comprise providing many types of alerts. One or more of a visual alert or an audio alert for a user or the patient may be provided. The visual alert may comprise a captioned alert. The one or more of the visual alert or the audio alert may indicate one or more of a low inspiratory air pressure, a high inspiratory air pressure, a low inspiratory oxygenation level, a high inspiratory oxygenation level, a low inspiratory air temperature, a high inspiratory air temperature, a battery level, or a system error.

The patient interface may comprise one or more of a nasal cannula, a nasal prong, a nasopharyngeal tube, an esophageal tube, a mouth piece, or a face mask.

According to further aspects of the present disclosure, an exemplary continuous positive airway pressure (CPAP) system may comprise an inspiratory portion coupled to a patient interface to provide an airflow with positive pressure to a patient through the patient interface, an expiratory portion coupled to the patient interface to receive air exhaled from the patient, a controller coupled to the inspiratory and expiratory portions to receive one or more sensor measurements therefrom, and a user interface comprising a display. The controller may be configured to cause the display to provide a visual alert to one or more of a user or the patient in response to the received one or more sensor measurements.

The user interface may further comprise an audio output. The controller may be further configured to cause the display to provide an audio alert to the one or more of the user or the patient in response to the received one or more sensor measurements.

The one or more of the visual alert or the audio alert may indicate one or more of a low inspiratory air pressure, a high inspiratory air pressure, a low inspiratory oxygenation level, a high inspiratory oxygenation level, a low inspiratory air temperature, a high inspiratory air temperature, a battery level, or a system error.

The visual alert may comprise a captioned alert.

The controller may be further configured to couple to one or more external sensors. The controller may be further configured to cause the display to provide the visual alert in response to one or more external sensor measurements. The one or more external sensors may be configured to measure or determine one or more of a blood oxygenation level of the patient, a blood carbon dioxide level of the patient, a respiratory rate of the patient, a temperature of the patient, a cyanosis level of the patient, a vocalization of the patient, a capillary refill rate of the patient, or an input from the user.

The patient interface may comprise a nasal cannula, a nasal prong, a nasopharyngeal tube, an esophageal tube, a mouth piece, or a face mask.

According to further aspects of the present disclosure, an exemplary method of continuous positive airway pressure therapy may be provided. An airflow with positive pressure may be provided to a patient. Air exhaled from the patient may be received. One or more sensor measurements of one or more of the provided airflow or the received air exhaled may be received. A visual alert to one or more of a user or the patient in response to the received one or more sensor measurement may be provided. An audio alert may be provided to the one or more of the user or the patient in response to the received one or more sensor measurements.

The one or more of the visual alert or the audio alert may indicate one or more of a low inspiratory air pressure, a high inspiratory air pressure, a low inspiratory oxygenation level, a high inspiratory oxygenation level, a low inspiratory air temperature, a high inspiratory air temperature, a battery level, or a system error. The visual alert may comprise a captioned alert.

One or more external sensor measurements may be received and the visual alert may be provided in response to the one or more external sensor measurements. The one or more external sensor measurements may comprise one or more of a blood oxygenation level of the patient, a blood carbon dioxide level of the patient, a respiratory rate of the patient, a temperature of the patient, a cyanosis level of the patient, a vocalization of the patient, a capillary refill rate of the patient, or an input from the user.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings (also “Figure” and “FIG.” herein), of which:

FIG. 1 is an exemplary schematic of a known CPAP system;

FIG. 2 is an exemplary schematic of an improved CPAP system, according to embodiments of the present disclosure;

FIGS. 3A-3B are exemplary schematics of inspiratory modules of the improved CPAP system, according to embodiments of the present disclosure;

FIG. 4 is an exemplary front view of an exemplary CPAP system according to embodiments of the present disclosure; and

FIG. 5 is an exemplary schematic of the air flow in the inspiratory and expiratory modules of the improved CPAP system, according to embodiments of the present disclosure.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Existing systems, devices, and methods for RD therapy include: Standalone CPAP, “Indigenous” or “Homemade” CPAP, High Flow Nasal Cannula (HFNC), and Ventilation/Ventilator.

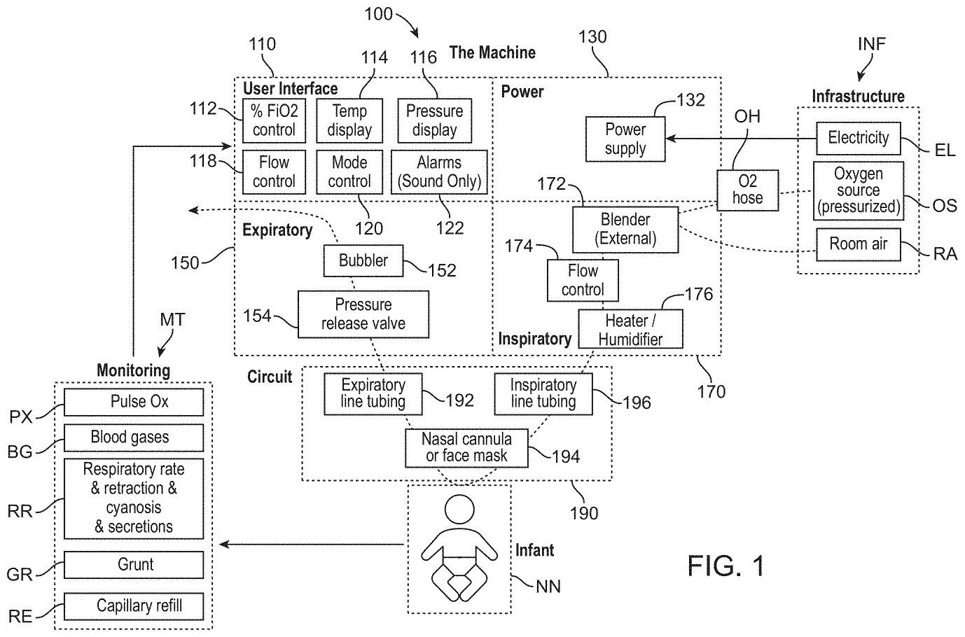

Standalone CPAP: CPAP is an advanced form of respiratory therapy that supplies a source of pressurized, heated, humidified, blended air to the patient, effectively holding the airways open to facilitate gas exchange. Because RDS is characterized by collapsing airways, CPAP is a well-suited mode of therapy that is widely accepted. FIG. 1 shows an exemplary CPAP system 100. As shown by in the FIG. 1, this method is flow rate driven, with a flow rate set at the inspiratory limb or portion 170 and pressure limited through the use of the bubbler. Additionally, standalone CPAP employs a combination of air and oxygen to ensure proper oxygen saturation while using the lowest amount of oxygen possible. Compared to oxygen therapy, CPAP more than doubles the RDS survival rate.

The exemplary CPAP system or machine 100 in FIG. 1 generally comprises a user interface 110, a power module 130, an expiratory portion 150, an inspiratory portion, and a patient circuit 190. The CPAP system can interface with medical infrastructure INF, including a source of electricity EL, an oxygen source OS delivering oxygen through an oxygen hose OH to the CPAP system 100, and room air RA. The neonate NN is concurrently monitored MT in a variety of ways, including pulse oximetry PX, measurement of blood gases BG such as oxygen and carbon dioxide, measurements of respiratory rate, retraction, cyanosis, secretions, etc. RR, monitoring of grunts GR, and monitoring of capillary refill RE.

The user interface 110 generally includes a display and/or control for the fraction of inspired oxygen (FiO₂) 112, a display and/or control for temperature 114, a display and/or control for pressure 116, and a display and/or control for flow 118 for air provided to the neonate NN. The user interface 110 generally further includes a mode controller 120 for the system 100 as well as audio alarms 122.

The power module 130 generally comprises a power supply 132 that may connect with the electricity source EL provided by the local medical infrastructure INF.

The inspiratory portion 170 draws air and/or oxygen from the medical infrastructure INF, particularly from the pressurized oxygen source OS and the room air RA. Compressed oxygen and ambient or room air are blended together by a blender 172 at the inspiratory portion 170. The blender 172 is typically an external unit and is controlled by a flow controller 174. The blended air is then directed to a heater and/or humidifier 176, before being directed to the patient interface circuit 190.

The patient interface circuit 190 includes an inspiratory line tubing 196, a patient interface 194, and an expiratory line tubing 192 leading to the expiratory portion 150. The patient interface 194 can be worn by the treated neonate NN and can be in the form of a nasal cannula(s), a nasal prong(s), a nasopharyngeal tube, an esophageal tube, a mouth piece, or a face mask.

The expiratory portion 150 receives expired air from the neonate NN and passes this air through a bubbler 152. The expiratory portion 150 can also include a pressure release valve 154 for the expired air.

“Indigenous” or “Homemade” CPAP: when standalone CPAPS are unable to be acquired or are economically feasible, indigenous CPAP may be employed. Using this method, oxygen is pressurized by putting one end of the tube under water (similar to the bubbler used in standalone CPAP). The indigenous CPAP approach is often 100% oxygen, which is either dry or passively humidified using a bottle of water. Similar to the delivery of low flow oxygen, it carries risks of oxygen toxicity such as blindness.

High Flow Nasal Cannula (HFNC): a newer modality of treatment—called High Flow Nasal Cannula (HFNC)—also delivers a source of heated, humidified, blended air to the patient. HFNC is less labor-intensive to deliver than CPAP, which is why it is gaining popularity in the neonatal community. However, it is difficult to know what pressure is being delivered, so HFNC is more often used as a step-down therapy than a primary treatment mode.

Ventilator: severe cases of respiratory distress, in which patients cannot initiate breaths, are treated with mechanical ventilation. Ventilators regulate breathing for the infant, rather than simply supplementing it. In low-resource areas, when hospitals are understaffed, or do not have the appropriate CPAP equipment infants are often unnecessarily escalated to ventilators which pose a higher risk than traditional CPAP.

Novel systems, devices, and methods for RD therapy according to embodiments of the present disclosure are now described.

The neonatal CPAP system proposed herein generally works via the same mechanisms as described for the CPAP system 100 above. It can provide heated, humidified, pressurized, blended air to the infant in order to maintain the airways and provide sufficient oxygenation. Additionally, there are several improvements that will decrease the burden on nurses, allowing more babies to receive this life-saving therapy and decreasing the number of cases that need to be escalated to more aggressive, higher-risk therapies.

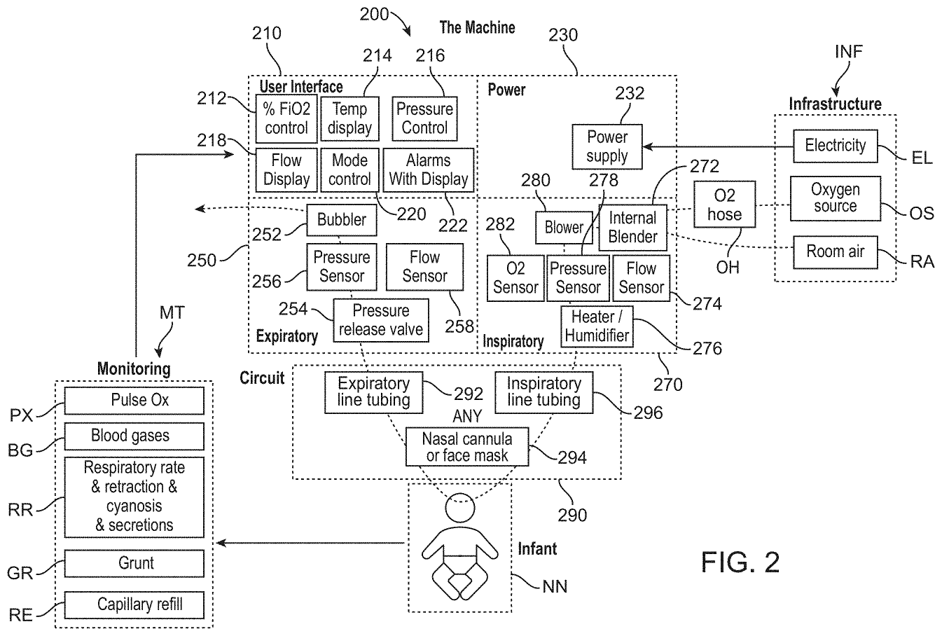

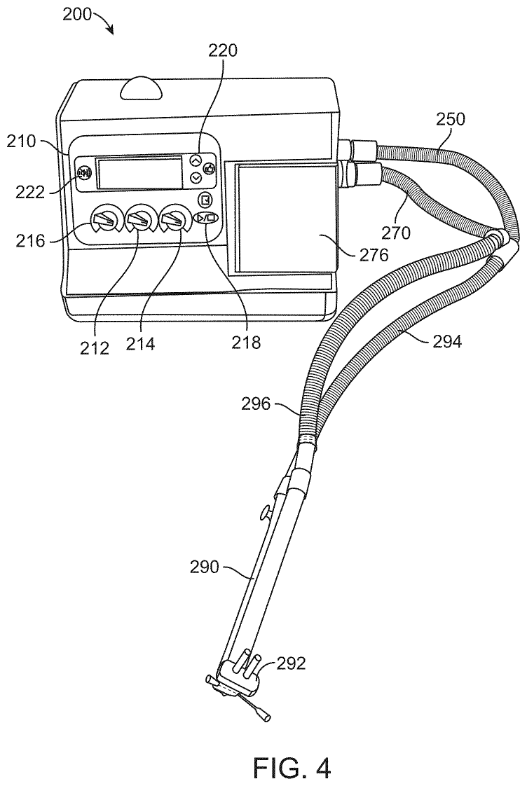

A comparative diagram/schematic between the traditional CPAP system 100 and the novel CPAP system 200 is provided in FIG. 2, and an example of the novel CPAP system 200 is shown in FIG. 4. The CPAP system or machine 200 may comprise many of the same components as the traditional CPAP 100. The CPAP system 200 may comprise a user interface 210, a power module 230, an expiratory portion 250, an inspiratory portion 270, and a patient circuit 290. The CPAP system can interface with medical infrastructure INF, including a source of electricity EL, an oxygen source OS delivering oxygen through an oxygen hose OH to the CPAP system 200, and room air RA. The neonate NN can be concurrently monitored MT in a variety of ways, including pulse oximetry PX, measurement of blood gases BG such as oxygen and carbon dioxide, measurements of respiratory rate, retraction, cyanosis, secretions, etc. RR, monitoring of grunts GR, and monitoring of capillary refill RE.

The user interface 210 generally includes a display and/or control for the fraction of inspired oxygen (FiO₂) 212, a display and/or control for temperature 214, a display and/or control for pressure 216, and a display and/or control for flow 218 for air provided to the neonate NN. The user interface 210 generally further includes a mode controller 220 for the system 200 as well as provides alarms or alerts 222. The alarms or alerts 222 can include both audio and visual alarms and/or alerts.

The power module 230 generally comprises a power supply 232 that may connect with the electricity source EL provided by the local medical infrastructure INF.

The inspiratory portion 270 may draw air and/or from the medical infrastructure INF, particularly from the pressurized oxygen source OS and the room air RA. The pressurized oxygen source OS may comprise one or more of wall oxygen, an oxygen tank, an oxygen concentrator, or a compressed oxygen line. Compressed oxygen and ambient or room air may be blended together by a blender 272 which is instead typically an internal unit and may be controlled by a flow sensor and controller 274. The blended air may have an oxygen concentration of between the oxygen concentration of the room air RA (typically about 21%) and the oxygen concentration of the compressed oxygen (typically 100%). The oxygenation concentration of the blended air may be determined with one or more oxygenation sensors, flow rate sensor, or pressure sensors as described further herein, and the blending ratio of room air RA and compressed oxygen may be varied, such as by user selection, to achieve a desired oxygen concentration.

The blended air may then be directed to a heater and/or humidifier 276 with a blower 280, before being directed to the patient interface circuit 290. The heater and/or humidifier 267 may heat and/or humidify the blended air so that it is comfortable and safe for the patient to breath. For example, the blended air may be heated to a temperature close to the normal body temperature of the body, such as between 34° C. and 41° C., preferably about 37° C. Also, for example, the blended air may be humidified to be at 100% humidity or less, 95% humidity or less, 90% humidity or less, 85% humidity or less, 80% humidity or less, 75% humidity or less, 70% humidity or less, 65% humidity or less, 60% humidity or less, 55% humidity or less, or 50% humidity or less. The blended air may be directed to the patient through the patient interface circuit 290 at a flow rate sufficient to provide adequate oxygen to the patient while minimizing injury risk, for example, a flow rate of between 3 and 15 L/min. A pressure sensor 278 and/or an oxygen sensor 282 may also be coupled to the air flow from the blower 280. The inspiratory portion 270 may be in the form of a standalone module.

The patient interface circuit 290 may include an inspiratory line tubing 296, a patient interface 294, and an expiratory line tubing 292 leading to the expiratory portion 250. The patient interface 294 can be worn by the treated neonate NN and can be in the form of a nasal cannula(s), a nasal prong(s), a nasopharyngeal tube, an esophageal tube, a mouth piece, a face mask, or any suitable patient interface.

The expiratory portion 250 may receive expired air from the neonate NN and may pass this expired air through a bubbler 252. The expiratory portion 250 can also include a pressure release valve 254 for the expired air. A pressure sensor 256 and/or an oxygen sensor 258 may also be coupled to the expired air directed to the bubbler 252. The expiratory portion 250 may be in the form of a standalone module.

### Key Device Features

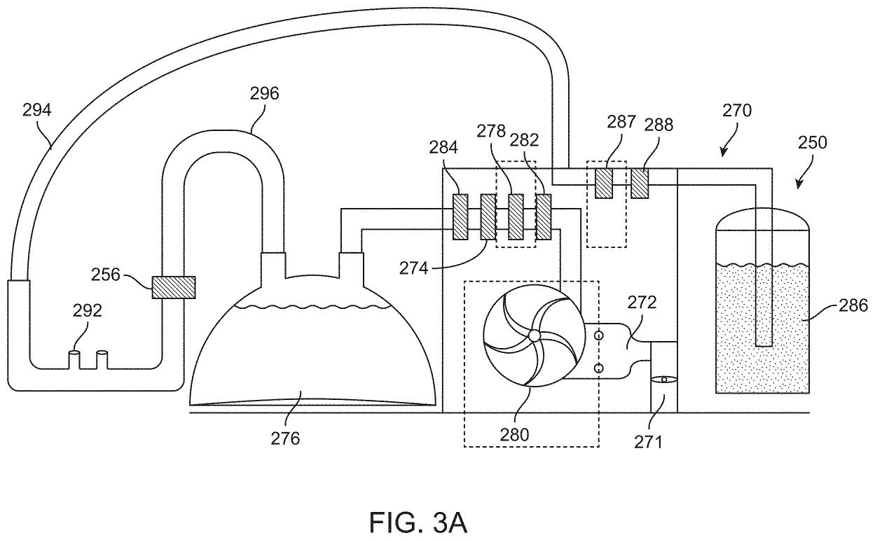

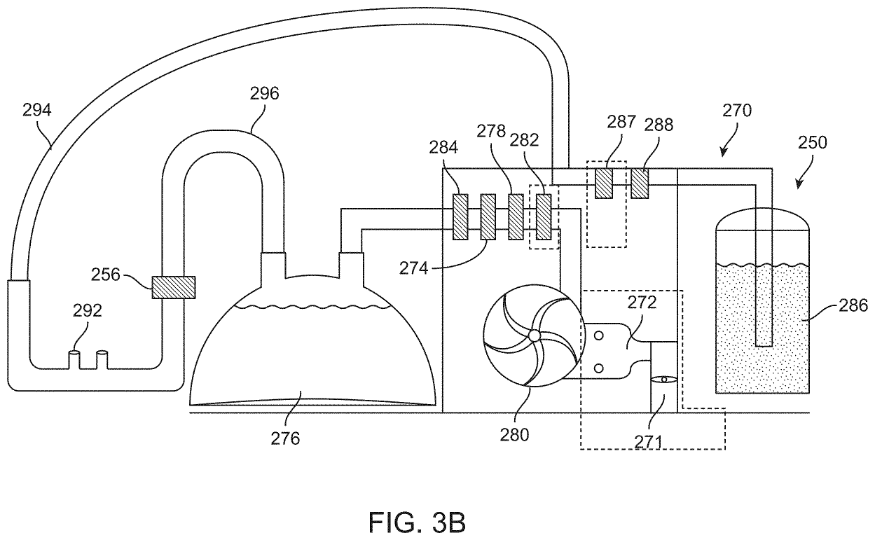

Blower-driven: the CPAP system 200 is intended to be pressure, rather than flow driven, and may generate pressure using a small, brushless blower 280. The pressure sensor(s) may be attached to both the inspiratory circuit 270 and expiratory circuit 250, allowing for a feedback loop that can trigger adjustment of the blower speed to maintain the target pressure set by the clinician. Using a blower 280 rather than a pneumatic system can eliminate the need for a source of compressed air (e.g., wall oxygen, oxygen tank, compressed air line). Compressed air may not available in many rural hospitals, and a blower is a smaller, quieter, and more cost-effective solution than a traditional compressor. Blower and control components are highlighted in FIG. 3A.

Blending and FiO₂ control: the blower 280 may pull room air RA into a mixing chamber through a vent in the housing of the device. If additional oxygen is required, any oxygen source OS (e.g., wall oxygen, oxygen canister, oxygen tank, oxygen concentrator, or compressed oxygen line) can be connected to the blender 272 where it will combine with room air RA and pushed into the blower 280. The target oxygen concentration may be set by the user and monitored by an oxygen sensing cell or sensor 282. Alternatively or in combination, the flow rates or pressures of the room air RA, compressed oxygen, and blended air may be used to determine the oxygen concentration of the blended air as described further herein. A proportional valve 271 placed between the oxygen source OS and the blender 272 may control the flow of oxygen to maintain the target FiO2 despite changes in flow rate. Many indigenous CPAP set-ups, as well as some bubble-CPAP systems, do not use a blender. This typically results in 100% oxygen delivered to the baby and increases the risk of retinopathy of prematurity (ROP). Those CPAPs that do call for blenders generally require a separate blender to be purchased. The CPAP system 200 will typically have a blender 280 integrated into the device itself. While current blenders can be electronic or mechanical, the proposed blender 272 may be electronic to allow integrated for FiO₂ automation at a lower cost than traditional mechanical options. Oxygen control components are highlighted in the FIG. 3B.

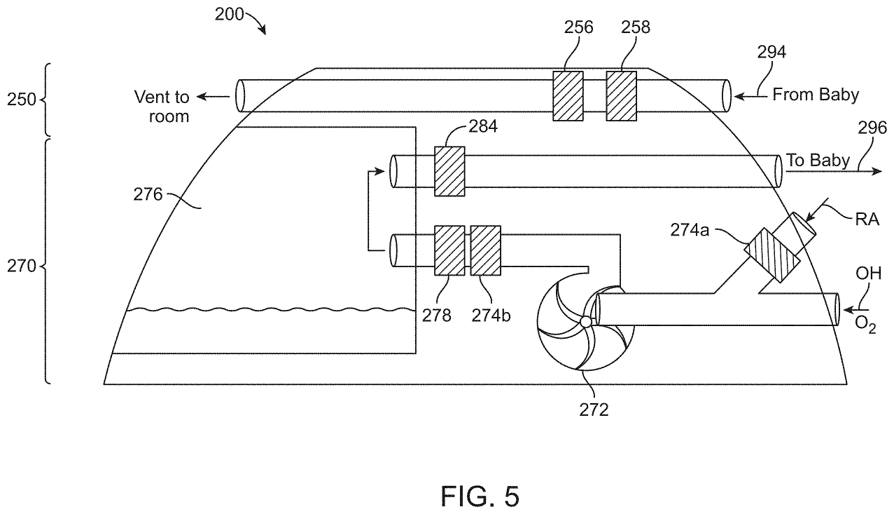

FIG. 5 shows a schematic of the airflow in the novel CPAP system 200. Starting at the inspiratory portion 270, room air RA and compressed oxygen OH may be taken in and blended with the blender 272. A flow rate sensor 274a may be provided at the intake of the room air RA to determine the flow rate of the room air RA taken into the system 200. Alternatively or in combination, a pressure sensor may be provided at the intake of the room air RA to determine the pressure of the room air RA taken into the system 200. The blended air may then be directed to the heater and/or humidifier 276. A flow rate sensor 274a and/or a pressure sensor 278 may be provided to determine the flow rate and/or pressure, respectively, of the blended air directed into the heater/humidifier 276.

The ratio of room air RA to compressed oxygen OH in the blended air, and therefore the oxygenation of the blended air (assuming the oxygenation of the room air RA (typically 21%) and the compressed oxygen (typically 100%) is known), may be determined based on the flow rate and/or pressure of the directed blended air as compared to the flow rate and/or pressure of the room air RA and/or compressed oxygen OH directed into the blender 272. For example, the flow rate of the blended air may equal the flow rate of the room air RA combined with the flow rate of the compressed oxygen OH directed into the blender 272 (as normalized based on the known cross-sectional area of the air flow, as appropriate), and by measuring the flow rate of the blended air with sensor 274b and the flow rate of the room air RA with the flow rate sensor 274a, the flow rate of the compressed oxygen OH can be determined, and the ratio of room air RA to compressed oxygen OH in the blended air can be determined, and the oxygenation of the blended air can be determined based on this ratio. Similar calculations may be made for pressure instead of flow rate, with the flow rate and/or pressure of the compressed oxygen OH directed into the blender 272 measured instead of that of the room air RA, and/or with the flow rates and/or pressures of the both the room air RA and the compressed oxygen OH directed into the blender 272 measured instead of that of the blended air. As discussed above, the determined oxygenation may be displayed to the user. The CPAP system 200 may allow a user or other medical professional to set the CPAP system 200 to have a desired oxygenation percentage in the blended air, for example, by varying the flow rates of the room air RA and the compressed oxygen OH.

Referring back to FIG. 5, the blended air may then be directed to the infant or neonate 262 after heating and/or humidification. A temperature sensor 284 may be provided to measure the heated and/or humidified blended air. Referring now to the expiratory portion 250, expired air from the infant and/or neonate NN may be vented to the room. A pressure sensor 256 and/or a flow rate sensor 258 may be provided to measure the pressure and/or flow rate, respectively, of the expired air.

Console-side detection and control: Many known CPAP devices rely on pressure and/or flow rate sensors at the patient interface (e.g., the nasal cannula(s), the nasal prong(s), the nasopharyngeal tube, the esophageal tube, the mouth piece, or the face mask) to detect air flow parameters so that the CPAP device can be controlled accordingly. The CPAP system or machine 200 may provide pressure/or flow rate sensors at the inspiratory circuit 270 and the expiratory circuity 250 of the CPAP system or machine 200 (i.e., at the console or system box itself). Measurements from the inspiratory circuit and expiratory circuit sensors may be used to determine air flow parameter(s) at the patient interface. The CPAP system or machine 200 may then be adjusted accordingly to adjust air flow parameter(s) at the patient interface to a desired level or levels. In doing so, the CPAP system or machine 200 can be advantageously workable with many types of patient interfaces, including those without any sensors. For optimal use of a particular user interface, the CPAP system or machine 200 may be calibrated for accurate determination of the air flow parameter(s) at the patient interface as further described herein.

Calibration/Automatic pressure control (leak compensation): unlike traditional CPAP systems that often require specific occluding (nasal) prongs or other specific user interfaces, the CPAP system 200 herein can is configured to be used with any brand of occluding or non-occluding nasal prongs or other user interfaces. This can be achieved via the use of pressure and flow sensors at both the inspiratory portion 270 and the expiratory portion 250, as well as a calibration sequence that is run prior to each use. During calibration, the blower 280 may be turned on to one or more known levels, and pressure and flow are measured in the system 200. A controller or processor of the CPAP system 200 can calculate the resistance of the circuits (inspiratory portion 270, expiratory portion 250, and the patient interface portion 290) and can then be able to calculate the pressure at the patient interface 294 based on the pressure and flow in the inspiratory portion 270 and the expiratory portion 250. If the pressure in the system 200 changes for any reason (for example, prongs becoming loose in the baby's nose), the sensors may measure the change in pressure, and the blower may alter its speed to compensate for the change in pressure. The system 200 may also monitor the flow rate to ensure that it never exceeds the target maximum flow.

This novel feature can provide a dual benefit: it can allows for any circuit to be used with the CPAP system 200 (not available with other standalone CPAPs) and can also decrease the need for nurse oversight as a slipped prong will no longer diminish the effectiveness of therapy or require excessive time to triage.

Captioned Alarms: current CPAP modalities will not work if the prongs slip from the baby's nose, but not all systems will alarm if this occurs. Additionally, those that do alarm do not explain why the system has failed, which requires the nurse to triage the entire system. The CPAP system 200 may incorporate both alarms and a screen that will typically display the cause of the alarm so the nurse can rapidly correct the problem and return to other work. This can decrease both the amount of time that a baby will spend without therapy and the amount of time a nurse spends with each infant on CPAP. Other alarms or alerts 222 that the user interface 210 may provide include those to indicate a low inspiratory air pressure, a high inspiratory air pressure, a low inspiratory oxygenation level, a high inspiratory oxygenation level, a low inspiratory air temperature, a high inspiratory air temperature, a battery level, or a system error.

While preferred embodiments of the present disclosure have been shown and described herein, it will be obvious to those skilled in the art that such embodiments are provided by way of example only. Numerous variations, changes, and substitutions will now occur to those skilled in the art without departing from the present disclosure. It should be understood that various alternatives to the embodiments of the present disclosure described herein may be employed in practicing the inventions of the present disclosure. It is intended that the following claims define the scope of the invention and that methods and structures within the scope of these claims and their equivalents be covered thereby.

What is claimed is:

1. A continuous positive airway pressure (CPAP) system comprising:
an inspiratory module comprising:
(i) a first inlet for a compressed oxygen source,
(ii) a second inlet for an ambient air source,
(iii) a blender coupled to the first and second inlets to blend compressed oxygen and ambient air from the first and second inlets, respectively,
(iv) a blower coupled to the blender and configured to generate an airflow directed to a patient from the blended air.

2. The system of claim 1, wherein the inspiratory module further comprises an outlet to couple to a patient interface and direct the airflow thereto.

3. The system of claim 2, wherein the patient interface comprises one or more of a nasal cannula, a nasal prong, a nasopharyngeal tube, an esophageal tube, a mouth piece, or a face mask.

4. The system of claim 1, wherein the inspiratory module further comprises a heater to provide heat to the airflow generated by the blower.

5. The system of claim 4, wherein the inspiratory module further comprises a temperature sensor to measure a temperature of the airflow.

6. The system of claim 5, wherein the inspiratory module further comprises a controller configured to control the amount of heat provided to the airflow by the heater based on the measured temperature.

7. The system of claim 1, wherein the inspiratory module further comprises a humidifier to provide humidity to the airflow generated by the blower.

8. The system of claim 7, wherein the inspiratory module further comprises a humidity sensor to measure a humidity of the airflow.

9. The system of claim 8, wherein the inspiratory module further comprises a controller configured to control the amount of humidity provided to the airflow by the humidifier based on the measured humidity.

10. The system of claim 1, wherein the inspiratory module further comprises a pressure sensor to measure a pressure of the airflow generated by the blower.

11. The system of claim 10, wherein the inspiratory module further comprises a controller configured to control the blower based on the measured pressure.

12. The system of claim 1, wherein the inspiratory module further comprises a flow rate sensor to measure a flow rate of the airflow generated by the blower.

13. The system of claim 12, wherein the inspiratory module further comprises a controller configured to control the blower based on the measured flow rate.

14. The system of claim 1, wherein the inspiratory module further comprises an oxygen sensor to measure an oxygenation of the blended air.

15. The system of claim 14, wherein the inspiratory module further comprises a controller configured to control a ratio of compressed air provided through the first inlet to ambient air provided by the second inlet based on the measured oxygenation.

16. The system of claim 1, wherein the inspiratory module comprises two or more of (i) a compressed oxygen sensor to measure one or more of a flow rate or pressure from the compressed air from the first inlet, (ii) an ambient air sensor to measure one or more of a flow rate or pressure from the ambient air from the second inlet, or (iii) a blended air sensor to measure one or more of a flow rate or pressure from the blended air from the blender, and wherein the inspiratory module further comprises a controller coupled to the two or more of (i) the compressed oxygen sensor, (ii) the ambient air sensor, or (iii) the blended air sensor to determine an oxygenation of the blended air based on the flow rates or pressures from two or more of (i) the compressed air, (ii) the ambient air, and (iii) the blended air.

17. The system of claim 16, wherein the controller is configured to control a ratio of compressed air provided through the first inlet to ambient air provided by the second inlet based on the determined oxygenation.

18. The system of claim 1, wherein the compressed air source comprises one or more of wall oxygen, an oxygen tank, or a compressed oxygen line.

19. A continuous positive airway pressure (CPAP) system comprising:
an inspiratory module comprising
(i) a first inlet for a compressed oxygen source,
(ii) a second inlet for an ambient air source,
(iii) a blender coupled to the first and second inlets to blend compressed oxygen and ambient air from the first and second inlets, respectively,
(iv) one or more sensors to determine an oxygenation of the blended air, and
(v) a controller configured to control a ratio of the compressed air provided through the first inlet to the ambient air provided by the second inlet based on the determined oxygenation,
wherein the ratio is controlled to maintain a desired range of oxygen concentration in the blended air.

20. The system of claim 19, wherein the inspiratory module further comprises a heater to provide heat to the blended air.

21. The system of claim 20, wherein the inspiratory module further comprises a temperature sensor to measure a temperature of the blended air.

22. The system of claim 21, wherein the inspiratory module further comprises a controller configured to control the amount of heat provided to the blended air by the heater based on the measured temperature.

23. The system of claim 19, wherein the inspiratory module further comprises a humidifier to provide humidity to the blended air.

24. The system of claim 23, wherein the inspiratory module further comprises a humidity sensor to measure a humidity of the blended air.

25. The system of claim 24, wherein the inspiratory module further comprises a controller configured to control the amount of humidity provided to the blended air by the humidifier based on the measured humidity.

26. The system of claim 19, wherein the compressed air source comprises one or more of wall oxygen, an oxygen tank, an oxygen concentrator, or a compressed oxygen line.

27. The system of claim 19, wherein the one or more sensors comprises an oxygen sensor.

28. The system of claim 19, wherein the one or more sensors comprise two or more of (i) a compressed oxygen sensor to measure one or more of a flow rate or pressure from the compressed air from the first inlet, (ii) an ambient air sensor to measure one or more of a flow rate or pressure from the ambient air from the second inlet, or (iii) a blended air sensor to measure one or more of a flow rate or pressure from the blended air from the blender, and wherein the inspiratory module further comprises a controller coupled to the two or more of the (i) compressed oxygen sensor, (ii) the ambient air sensor, and (iii) the blended air sensor to determine an oxygenation of the blended air based on the flow rates or pressures from two or more of (i) the compressed air, (ii) the ambient air, and (iii) the blended air.

29. A method of generating an airflow for continuous positive airway pressure therapy, the method comprising:
providing ambient air and compressed oxygen to a blender to generate blended air;
generating an airflow of the blended air with a blower;
determining an oxygenation of the blended air; and
controlling a ratio of the compressed oxygen to the ambient air provided to the blender based on the determined oxygenation,
wherein the ratio is controlled to maintain a desired range of oxygen concentration in the blended air.

30. The method of claim 29, further comprising heating the blended air.

31. The method of claim 30, further comprising measuring a temperature of the blended air and controlling the amount of heat provided to the blended air based on the measured temperature.

32. The method of claim 29, further comprising humidifying the blended air.

33. The method of claim 32, further comprising measuring a humidity of the blended air and controlling the humidity provided to the blended air based on the measured humidity.

34. The method of claim 29, further comprising measuring one or more of a flow rate or a pressure of the airflow generated by the blower controlling one or more of a compressed air source and providing compressed air to the blender, the blender, or the blower in response.

35. The method of claim 29, wherein the compressed air is provided from one or more of wall oxygen, an oxygen tank, an oxygen concentrator, or a compressed oxygen line.

36. The method of claim 29, wherein the oxygenation of the blended air is determined with an oxygenation sensor.

37. The method of claim 29, wherein the oxygenation of the blended air is determined based on one or more of a flow rate or pressure from the compressed oxygen, one or more of a flow rate or pressure from the ambient air, and one or more of a flow rate or pressure from the blended air.

38. A continuous positive airway pressure (CPAP) system comprising:
an inspiratory portion coupled to a patient interface to provide an airflow with positive pressure to a patient through the patient interface, the inspiratory portion including a first sensor to measure one or more of a pressure or a flow rate of the airflow at the inspiratory portion;
an expiratory portion coupled to the patient interface to receive air exhaled from the patient, the expiratory portion including a second sensor for measuring one or more of a pressure or a flow rate of the air exhaled at the expiratory portion; and
a controller configured to (i) determine a pressure at the patient interface based on the measured one or more of the pressure or the flow rate of the airflow at the inspiratory portion and the measured one or more of the pressure or the flow rate of the air exhaled at the expiratory portion and (ii) modify the airflow provided by the inspiratory portion based on the determined pressure at the patient interface.

39. The system of claim 38, wherein the inspiratory portion comprises a blower to generate the airflow, and wherein the controller is configured to modify the airflow by regulating a speed of the blower.

40. The system of claim 38, wherein the controller is configured to increase the airflow if the determined pressure has decreased.

41. The system of claim 38, wherein the inspiratory portion comprises a heater to provide heat to the airflow and a temperature sensor to measure a temperature of the airflow, and wherein the controller is configured to control the amount of heat provided to the airflow by the heater based on the measured temperature.

42. The system of claim 38, wherein the inspiratory portion comprises a humidifier to provide humidity to the airflow and a humidity sensor to measure humidity of the airflow, and wherein the controller is configured to control the amount of humidity provided to the airflow by the humidifier based on the measured humidity.

43. The system of claim 38, wherein the inspiratory portion comprises a blender to blend ambient air with compressed oxygen and one or more sensors to determine oxygenation of the airflow, and wherein the controller is configured to control a ratio of compressed air to ambient air based on the determined oxygenation.

44. The system of claim 38, wherein the inspiratory portion further comprises one or more of a blower to generate the airflow, a heater for the airflow, a humidifier for the airflow, or a blender to blend ambient air with compressed air.

45. The system of claim 38, wherein the inspiratory portion further comprises one or more of a flow rate sensor, a pressure sensor, a temperature sensor, a humidity sensor, or an oxygenation sensor.

46. The system of claim 38, wherein the inspiratory portion is removably coupled to the patient interface.

47. The system of claim 38, wherein the inspiratory portion is a standalone module.

48. The system of claim 38, wherein the expiratory portion further comprises an air bubbler for the air exhaled.

49. The system of claim 38, wherein the expiratory portion is removably coupled to the patient interface.

50. The system of claim 38, wherein the expiratory portion is a standalone module.

51. The system of claim 38, further comprising a user interface coupled to the controller.

52. The system of claim 51, wherein the user interface comprises a visual display.

53. The system of claim 52, wherein the visual display is configured to display one or more of the measured pressure of the airflow at the inspiratory portion, the measured flow rate of the airflow at the inspiratory portion, the measured pressure of the airflow at the expiratory portion, the measured flow rate of the airflow at the expiratory portion, the determined pressure at the patient interface, or a calibration status of the system.

54. The system of claim 53, wherein the visual display is further configured to display one or more of a measured temperature, a measured humidity, or a measured oxygenation level of the airflow at the inspiratory portion.

55. The system of claim 52, wherein the visual display is configured to provide a visual alert for a user or the patient.

56. The system of claim 55, wherein the visual alert comprises a captioned alert.

57. The system of claim 51, wherein the user interface is configured to provide one or more of a visual alert or an audio alert for a user or the patient.

58. The system of claim 57, wherein the one or more of the visual alert or the audio alert indicates one or more of a low inspiratory air pressure, a high inspiratory air pressure, a low inspiratory oxygenation level, a high inspiratory oxygenation level, a low inspiratory air temperature, a high inspiratory air temperature, a battery level, or a system error.

59. The system of claim 38, wherein the patient interface comprises one or more of a nasal cannula, a nasal prong, a nasopharyngeal tube, an esophageal tube, a mouth piece, or a face mask.

60. A method of generating an airflow for continuous positive airway pressure therapy, the method comprising:
measuring one or more of a pressure or a flow rate of an airflow generated at an inspiratory portion of a continuous positive airway pressure (CPAP) system;
measuring one or more of a pressure or a flow rate of air exhaled and received at an expiratory portion of the CPAP system;
determining a pressure at a patient interface coupled to the CPAP system based on the measured one or more of the pressure or the flow rate of the airflow at the inspiratory portion and the measured one or more of the pressure or the flow rate of the air exhaled at the expiratory portion; and
modifying the airflow provided by the inspiratory portion based on the determined pressure at the patient interface.

61. The method of claim 60, wherein modifying the airflow comprises regulating a speed of a blower of the inspiratory portion of the CPAP system generating the airflow.

62. The method of claim 60, wherein modifying the airflow comprises increasing the airflow if the determined pressure has decreased.

63. The method of claim 60, further comprising measuring a temperature of the airflow and controlling an amount of heat provided to the airflow by a heater in response.

64. The method of claim 60, further comprising measuring a humidity of the airflow and controlling an amount of humidity provided to the airflow by a humidifier in response.

65. The method of claim 60, further comprising determining an oxygenation of the airflow and controlling a ratio of compressed oxygen to ambient air blended by a blender of the inspiratory portion of the CPAP system based on the determined oxygenation.

66. The method of claim 60, further comprising directing the airflow to a patient through a patient interface coupled to the CPAP system.

67. The method of claim 60, further comprising displaying one or more of the measured pressure of the airflow at the inspiratory portion, the measured flow rate of the airflow at the inspiratory portion, the measured pressure of the airflow at the expiratory portion, the measured flow rate of the airflow at the expiratory portion, the determined pressure at the patient interface, or a calibration status of the system.

68. The method of claim 60, further comprising displaying one or more of a measured temperature, a measured humidity, or a measured oxygenation level of the airflow at the inspiratory portion.

69. The method of claim 60, further comprising providing one or more of a visual alert or an audio alert for a user or the patient.

70. The method of claim 69, wherein the visual alert comprises a captioned alert.

71. The method of claim 69, wherein the one or more of the visual alert or the audio alert indicates one or more of a low inspiratory air pressure, a high inspiratory air pressure, a low inspiratory oxygenation level, a high inspiratory oxygenation level, a low inspiratory air temperature, a high inspiratory air temperature, a battery level, or a system error.

72. The method of claim 60, wherein the patient interface comprises one or more of a nasal cannula, a nasal prong, a nasopharyngeal tube, an esophageal tube, a mouth piece, or a face mask.

73. A continuous positive airway pressure (CPAP) system comprising:
an inspiratory portion coupled to a patient interface to provide an airflow with positive pressure to a patient through the patient interface;
an expiratory portion coupled to the patient interface to receive air exhaled from the patient;
a controller coupled to the inspiratory and expiratory portions to receive one or more sensor measurements therefrom; and
a user interface comprising a display,
wherein the controller is configured to cause the display to provide a visual alert to one or more of a user or the patient in response to the received one or more sensor measurements.

74. The system of claim 73, wherein the user interface further comprises an audio output, and wherein the controller is further configured to cause the display to provide an audio alert to the one or more of the user or the patient in response to the received one or more sensor measurements.

75. The system of claim 73, wherein the one or more of the visual alert or the audio alert indicates one or more of a low inspiratory air pressure, a high inspiratory air pressure, a low inspiratory oxygenation level, a high inspiratory oxygenation level, a low inspiratory air temperature, a high inspiratory air temperature, a battery level, or a system error.

76. The system of claim 73, wherein the visual alert comprises a captioned alert.

77. The system of claim 73, wherein the controller is further configured to couple to one or more external sensors, and wherein the controller is further configured to cause the display to provide the visual alert in response to one or more external sensor measurements.

78. The system of claim 77, wherein the one or more external sensors are configured to measure or determine one or more of a blood oxygenation level of the patient, a blood carbon dioxide level of the patient, a respiratory rate of the patient, a temperature of the patient, a cyanosis level of the patient, a vocalization of the patient, a capillary refill rate of the patient, or an input from the user.

79. The system of claim 73, wherein the patient interface comprises a nasal cannula, a nasal prong, a nasopharyngeal tube, an esophageal tube, a mouth piece, or a face mask.

80. A method of continuous positive airway pressure therapy, the method comprising:
providing an airflow with positive pressure to a patient;
receiving air exhaled from the patient;
receiving one or more sensor measurements of one or more of the provided airflow or the received air exhaled;
providing a visual alert to one or more of a user or the patient in response to the received one or more sensor measurements.

81. The method of claim 80, further comprising providing an audio alert to the one or more of the user or the patient in response to the received one or more sensor measurements.

82. The method of claim 80, wherein the one or more of the visual alert or the audio alert indicates one or more of a low inspiratory air pressure, a high inspiratory air pressure, a low inspiratory oxygenation level, a high inspiratory oxygenation level, a low inspiratory air temperature, a high inspiratory air temperature, a battery level, or a system error.

83. The method of claim 80, wherein the visual alert comprises a captioned alert.

84. The method of claim 80, further comprising receiving one or more external sensor measurements and providing the visual alert in response to the one or more external sensor measurements.

85. The method of claim 84, wherein the one or more external sensor measurements comprise one or more of a blood oxygenation level of the patient, a blood carbon dioxide level of the patient, a respiratory rate of the patient, a temperature of the patient, a cyanosis level of the patient, a vocalization of the patient, a capillary refill rate of the patient, or an input from the user.
